(19) United States
(12) Patent Application Publication
(10) Pub. No.: US 2006/0205938 A1
(43) Pub. Date: Sep. 14, 2006

(54) Monohydrate solvates of loracarbef

(76) Inventors: Yatendra Kumar, Haryana (IN); Neera Tewari, Haryana (IN); Hashim Nizar Poovanathi Nagoor Meeran, Kerla (IN); Bishwa Prakash Rai, Uttar Pradesh (IN); Sailendra Kumar Singh, Haryana (IN)

Correspondence Address: RANBAXY INC., 600 COLLEGE ROAD EAST, SUITE 2100, PRINCETON, NJ, 08540, US

(21) Appl. No.: 10/535,677

(22) PCT Filed: Nov. 21, 2003
(86) PCT No.: PCT/IB03/05331

§ 371 Date: Mar. 28, 2006

(30) Foreign Application Priority Data
Nov. 21, 2002 (IN) 1176/DEL/2002
Dec. 11, 2002 (IN) 1239/DEL/2002

(51) Int. Cl. C07D 471/02

(52) U.S. Cl. 540/205

(57) ABSTRACT

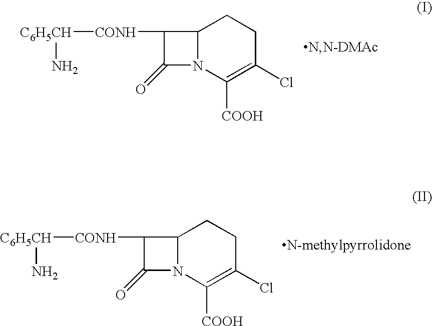

This invention relates to monohydrate solvates of loracarbef. The invention also relates to processes for preparing solvates of loracarbef, crystalline monohydrate of loracarbef from said solvates and pharmaceutical compositions that include the crystalline monohydrate of loracarbef.

### FIELD OF THE INVENTION

The field of the invention relates to monohydrate solvates of loracarbef. The invention also relates to processes for preparing solvates of loracarbef, crystalline monohydrate of loracarbef from said solvates and pharmaceutical compositions that include the crystalline monohydrate of loracarbef.

### BACKGROUND OF THE INVENTION

Loracarbef is a synthetic β-lactam antibiotic of the carbacephem class for oral administration. It is disclosed in U.S. Pat. No. 4,335,211. Chemically, loracarbef is (6R,7S)-7-[(R)-2-amino-2-phenylacetamido]-3-chloro-8-oxo-1-azabicyclo [4.2.0]oct-2-ene-carboxylic acid, monohydrate and has structural Formula I.

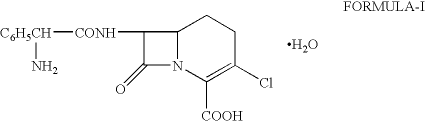

Loracarbef has shown activity against a broad spectrum of bacteria in laboratory tests. Loracarbef has proven to be a relatively stable compound, which exhibits high blood levels and relatively long half-life.

Loracarbef has been isolated in various forms, including the crystalline monohydrate form which is disclosed in the European Patent Publication, EP 0,311,366. The crystalline dihydrate form of loracarbef is disclosed in European Patent Publication, EP 0,369,686. Other known solvate forms of the compounds are bis (DMF), dihydrate mono(DMF) and mono (DMF) forms and are disclosed in U.S. Pat. No. 4,977,257. U.S. Pat. No. 5,580,977 discloses the crystalline anhydrate form of loracarbef.

Various solvates described above are convenient intermediates for preparing loracarbef, in general and the monohydrate form of loracarbef, in particular. It is well known that a compound intended for pharmaceutical use is desired to have sufficient density in order to facilitate the formulation of the bulk product. However, the process disclosed in EP 0,369,686 yields loracarbef monohydrate in the form of a fine, fluffy powder with a density of approximately 0.2 g/ml. This density renders the bulk product, loracarbef monohydrate, very difficult to formulate.

Accordingly, methods for the total synthesis of these promising compounds and intermediates to these compounds are highly desirable, particularly the methods, which are adaptable to large scale manufacture, and result in high yields and reduced cost of manufacture.

### SUMMARY OF THE INVENTION

In one general aspect there is provided a mono N, N-dimethylacetamide monohydrate solvate of loracarbef of Formula II-A.

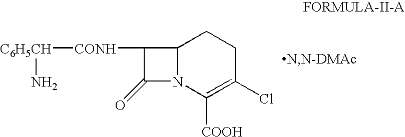

In another general aspect there is provided a mono N-methylpyrrolidone monohydrate solvate of loracarbef of Formula II-B.

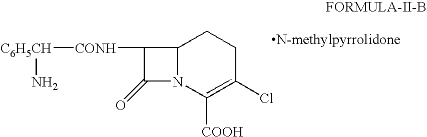

In another general aspect there are provided processes for the preparation of the mono N, N-dimethylacetamide monohydrate and mono N-methylpyrrolidone monohydrate solvates of loracarbef.

In another general aspect there is provided a process for the preparation of the crystalline monohydrate of loracarbef of Formula I from mono N, N-dimethylacetamide monohydrate solvate or mono N-methylpyrrolidone monohydrate solvate of loracarbef.

In another general aspect there is provided a crystalline monohydrate of loracarbef having a bulk density greater than or equal to 0.6 gm/ml.

In another general aspect there is provided a pharmaceutical composition that includes a therapeutically effective amount of a crystalline monohydrate of loracarbef having a bulk density greater than or equal to 0.6 gm/ml; and one or more pharmaceutically acceptable carriers, excipients or diluents.

### DETAILED DESCRIPTION OF THE INVENTION

The inventors have developed new monohydrate solvates of loracarbef, and in particular, the mono N, N-dimethylacetamide monohydrate solvate of Formula II-A and mono N-methylpyrrolidone monohydrate solvates of loracarbef of Formula II-B.

The mono N, N-dimethylacetamide monohydrate solvate is characterized by the X-ray powder diffraction pattern below:

| d | I/Io |
|---|---|
| 15.6 | 17.0 |
| 11.80 | 100 |
| 11.12 | 41 |
| 7.43 | 25 |
| 5.91 | 12 |
| 5.19 | 14 |
| 4.88 | 16 |
| 4.76 | 22 |
| 4.69 | 17 |
| 4.45 | 13 |
| 4.28 | 13 |
| 3.93 | 70 |
| 3.639 | 28 |
| 3.33 | 18 |
| 3.177 | 71 |
| 2.949 | 18 |
| 2.729 | 13 |
| 2.6122 | 13 |

The mono N-methylpyrrolidone monohydrate solvate is characterized by the X-ray powder diffraction pattern below:

| d | I/Io |
|---|---|
| 15.8248 | 14 |
| 15.2251 | 13 |
| 12.0338 | 100 |
| 8.0954 | 8 |
| 7.5189 | 33 |
| 5.9968 | 13 |
| 5.4668 | 12 |
| 5.3810 | 14 |
| 5.2605 | 18 |
| 4.8863 | 22 |
| 4.7513 | 37 |
| 4.4579 | 21 |
| 4.2997 | 22 |
| 4.1411 | 16 |
| 3.9939 | 55 |
| 3.6421 | 38 |
| 3.3858 | 18 |
| 2.7314 | 15 |

The diffraction patterns above were obtained on a Rigaku (RINT 2000) instrument with nickel-filtered copper radiation (Cu:Ni) of wavelength lambda.=1.5406 Angstrom. The interplanar spacings are in the column marked “d” and are in Angstroms and the relative intensities are in the column marked “I/I₀”.

The inventors also have developed processes for the preparation of the mono N, N-dimethylacetamide monohydrate and mono N-methylpyrrolidone monohydrate solvates of loracarbef. The inventors also have developed a process for the preparation of a crystalline monohydrate of loracarbef of Formula I from mono N, N-dimethylacetamide monohydrate and mono N-methylpyrrolidone monohydrate solvates of loracarbef. The resulting crystalline monohydrate of loracarbef has a bulk density greater than or equal to 0.6 gm/ml. The inventors also have developed pharmaceutical compositions that contain the crystalline monohydrate of loracarbef having a bulk density greater than or equal to 0.6 gm/ml, in admixture with one or more solid or liquid pharmaceutical diluents, carriers, and/or excipients.

In one aspect, the mono-N, N-dimethylacetamide monohydrate solvate of loracarbef of Formula II-A is prepared by a process comprising mixing a compound of Formula III, 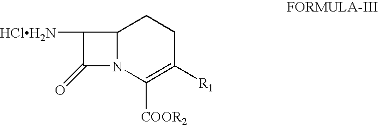
wherein R₁ is hydrogen, trihalo (C₁-C₄ alkyl) , C₁-C₄ alky, Cl-C₄ substituted alkyl, Cl-C₄ alkoxy, C₁-C₄ substituted alkoxy, C₁-C₆ alkylthio, C₁-C₆ substituted alkylthio, methoxy methyl, carbamoyloxy methyl, acetoxymethyl, C₂-C₆ alkenyl, C₂-C₆ substituted alkenyl, or halogen such as bromo, chloro, fluoro, and iodo; and R₂ is a carboxy-protecting group, with N,N, dimethylacetamide and a cyclic amine base containing 0-1 oxygen atoms or dimethylbenzylamine, to form a free amine of the compound of Formula IV, 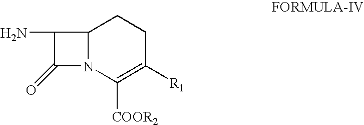
and reacting the free amine with an acylating agent of Formula V, 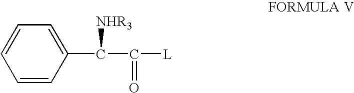
wherein R₃ is an amino protecting group and L is a leaving group.

In another aspect, the mono N-methylpyrrolidone monohydrate solvate of loracarbef of Formula II-B is prepared by a process comprising mixing a compound of Formula III, 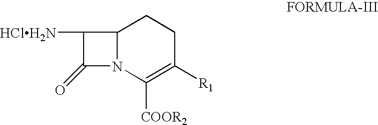
wherein R₁ is hydrogen, trihalo (C₁-C₄ alkyl) , C₁-C₄ alkyl, C₁-C₄ substituted alkyl, C₁-C₄ alkoxy, C₁-C₄ substituted alkoxy, C₁-C₆ alkylthio, C₁-C₆ substituted alkylthio, methoxy methyl, carbamoyloxy methyl, acetoxymethyl, C₂-C₆ alkenyl, C₂-C₆ substituted alkenyl, or halogen such as bromo, chloro, fluoro, and iodo; and R₂ is a carboxy-protecting group, with N-methylpyrrolidone and a cyclic amine base containing 0-1 oxygen atoms or dimethylbenzylamine, to form a free amine of the compound of Formula IV, 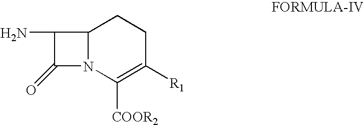
and reacting the free amine with an acylating agent of Formula V,
wherein R₃ is an amino protecting group and L is a leaving group.

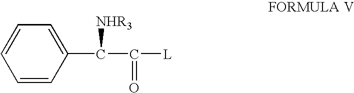

The term “carboxy-protecting group” refers to one of the ester derivatives of the carboxylic acid group which are not sterically hindered and are commonly employed to block or protect the carboxylic acid group while reactions are carried out on other functional groups of the compound. Examples of such groups include allyl, alkyl, benzyl, substituted benzyl groups, silyl group and halo-substituted alkyl groups, such as 2,2,2-trichloroethyl, 2,2,2-tribromoethyl, and 2-iodoethyl groups. Other examples of these groups include such as those found in E. Haslam, “Protective Groups in organic Chemistry”, J. G. W. McOmie, Ed., Plenum Press, New York, N.Y., 1973, Chapter 5, and T. W. Greene, “Protective Groups in Organic Synthesis”, John Wiley and Sons, New York, N.Y., 1981, Chapter 5. In particular, the carboxy-protecting group is 4-nitrobenzyl group.

The term “amino-protecting group” refers to substituents of the amino group commonly employed to block or protect the amino functionality while reactions are carried out on other functional groups of the compound.

The amino protecting group, R₃ includes carbamates, for example t-butoxycarbonyl or benzyloxycarbonyl, or the enamines. In particular, the amino-protecting groups include t-butoxycarbonyl, phenoxyacetyl, and enamines derived from (C₁-C₄ alkyl)acetoacetate groups. Other amino-protecting groups used in the cephalosporin, penicillin and peptide art are also embraced by the above terms. Further examples of groups referred to by the above terms are described by J. W. Barton, “Protective Groups in Organic Chemistry”, J. G. W. McOmie, Ed., Plenum Press, New York, N.Y., 1973, Chapter 2, and T. W. Greene, “Protective Groups in organic Synthesis”, John Wiley and Sons, New York, N.Y., 1981, Chapter 7.

The term “leaving group” means a leaving group which, under the reaction conditions will leave, thus allowing the free amine to bond to the carbonyl group. The leaving groups include those where L is of the Formula VI, 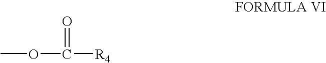
where R₄ is C₁-C₆ alkyl, or L is Cl, Br, I, active esters such as p-nitrophenyl; or the adducts of dicyclohexylcarbodiimide.

The base includes those consisting of five- or six- membered tertiary cyclic amines which may contain an oxygen atom, or dimethylbenzylamine. In particular, the tertiary cyclic amine bases include N-methylmorpholine (NMM) and N-methylpiperidine (NMP). The base can be used in an amount ranging from about 1 to about 1.3 molar equivalents, for example about 1.13 molar equivalents.

The hydrochloride salt of Formula III can be prepared by the process described in European Patent Application 0,266,896.

In general, the amino- and carboxy-protecting groups can be removed by methods well known in the art. Examples include such as those found in standard works on the subject, such as E. Haslam, “Protective Groups in Organic Chemistry”, J. G. W. McOmie, Ed., Plenum Press, New York, N.Y., 1973, Chapters 2 and 5, and T. W. Greene, “Protective Groups in Organic Synthesis”, John Wiley and Sons, New York, N.Y., 1981, Chapters 5 and 7, respectively.

For deprotecting the protected amino and protected carboxy groups, a mixture of concentrated HCl in water (2:1) is added to the acylation solution over a 30-45 minutes period at a temperature at 0° to −10° C. Zinc dust (about 3.5 equivalents) is then added over about 50-70 minutes, keeping the temperature at below 0° C. Approximately 1.2 equivalents of HCl is added and the reaction mixture is warmed to ambient temperature over about 45-60 minutes period. The mixture is stirred for about 5-6 hours at ambient temperature and semicarbazide hydrochloride (1.15 equivalents) is added, followed by 30-60 minutes of stirring. The pH is adjusted to about 2.9-3.1 with 28% aqueous ammonia and the mixture is filtered through a celite bed. The filtrate is warmed to 48-55° C. and is adjusted to a pH of 4.8 to 5.0 using 28% aqueous ammonia. The separated solid is further stirred for 30 minutes, and the pH is continuously adjusted to 5.8-6.2. The temperature of the mixture is lowered to 20-25° C. and a polar solvent is added, for example acetone and it is further stirred for another 30 minutes. The crystals are collected by filtration, washed with acetone, cooled to 20 -25° C., and dried to give the mono N, N-dimethylacetamide monohydrate or mono N-methylpyrrolidone monohydrate solvate of loracarbef.

In another aspect, the mono N, N-dimethylacetamide monohydrate solvate and mono N-methylpyrrolidone monohydrate solvate of loracarbef are converted to crystalline monohydrate of loracarbef. The loracarbef monohydrate prepared from the mono N, N-dimethylacetamide monohydrate solvate or mono N-methylpyrrolidone monohydrate solvate of loracarbef is found to have a bulk density equal to or greater than 0.6 g/ml.

In general, the monohydrate of loracarbef is prepared by suspending the mono N, N-dimethylacetamide monohydrate solvate or mono N-methylpyrrolidone monohydrate solvate of loracarbef in water. A clear solution of the starting material can be obtained by the addition of a minimum amount of acid, generally 6N (or more dilute) hydrochloric acid. The temperature of the solution is raised to about 50° C. followed by the slow addition of 28% ammonia solution to the solution until a pH of approximately 4.8 is obtained. The gradually developing suspension is stirred and maintained at about 50° C. during the addition of the base. The warm pH-adjusted suspension (50° C.) is cooled to approximately 20° C., stirred, filtered and the collected solid is dried at 40-45° C. to yield crystalline loracarbef monohydrate having bulk density equal to or greater than 0.6 g/ml.

The resulting crystalline monohydrate of loracarbef having a bulk density equal to or greater than 0.6 g/ml may be formulated into ordinary dosage forms such as, for example, tablets, capsules, pills, solutions, etc. In these cases, the medicaments can be prepared by conventional methods with conventional pharmaceutical excipients.

The present invention is further illustrated by the following examples which are provided merely to be exemplary of the invention and are not intended to limit the scope of the invention. Although the examples are directed to the mono N, N-dimethylacetamide monohydrate solvate and mono N-methylpyrrolidone monohydrate solvates of loracarbef, and crystalline monohydrate of loracarbef, the principles described in these examples can be applied to other solvates of loracarbef.

In the following Examples, the terms N, N-dimethylacetamide monohydrate solvate of loracarbef, nuclear magnetic resonance spectra, mass spectrum and infrared spectroscopy are abbreviated N,N-DMAc, NMR, MS and IR, respectively. In conjunction with the NMR spectra, the following abbreviations are used: “s” is singlet, “d” is doublet, “t” is triplet, “q” is quartet, and “m” is multiplet.

The NMR spectra were obtained on a Bruker (DRX 300) 300 MHz instrument. The chemical shifts are expressed in ppm values (parts per million downfield from tetramethylsilane).

### EXAMPLE 1

Preparation of Mono N,N-Dimethylacetamide Solvate of Loracarbef

Step A: Preparation of N-Methylmorpholine Salt

To a mixture of N, N-dimethylacetamide (60 ml) and N-methylmorpholine (3.0 g), p-nitrobenzyl 7 β-amino-3-chloro-1-carba (1-dethia)-3-cephem-4-carboxylic acid hydrochloride (10.0 g) was added in portions at 20-25° C. to form a free amine. The reaction mixture was stirred for 30 minutes and then cooled to −5 to −10° C.

Step B: Preparation of Mixed Anhydride

The Na/K Dane salt of phenylglycine, 9.3 g (prepared according to the procedure of Dane et al., Angew. Chem., Vol. 74, 873, 1962) was suspended in N, N-dimethylacetamide (150ml) and stirred for 30-40 minutes. The reaction mixture was cooled to −20 to −15° C. and methane sulphonic acid (0.12 g) and N-methylmorpholine (0.06 g) were added to it. Ethylchloroformate (3.3 g) was further added in one portion and stirring was continued for 90 minutes. at −10 to −15° C.

Step C: Condensation:

N-methylmorpholine hydrochloride solution containing the free amine obtained from Step A was slowly added to the mixed anhydride obtained from Step B at −20 to −10° C. The reaction mixture was stirred for 2.0 hours and the progress of the reaction was monitored by T.L.C. or HPLC. After completion of the reaction, a mixture of conc. HCl in H₂O (28 ml in 14 ml H₂O) was added over a 10-15 minutes period to diprotected loracarbef followed by the addition of zinc powder (6.0 g) and maintaining the temperature less than +5° C. The temperature was raised to 20-25° C. and the reaction mixture was stirred for about 2 hours. Semicarbazide hydrochloride (3.3 g) was added and the stirring was continued for 30-35 minutes. The pH of the reaction mixture was adjusted to 2.9 to 3.0 with 28% ammonia solution and then filtered it. The filtrate was warmed to about 48-55° C. and the pH was adjusted to 4.8 to 5.0 The separated solid was further stirred for 30 minutes and the pH was finally adjusted 5.8 to 6.2. The reaction mixture was cooled to 20-25° C., acetone was added, and stirred for another 30 minutes. It was then filtered and washed with acetone. The solid was dried under vacuum at 40-42° C. to give mono N,N-DMAc monohydrate solvate of loracarbef which was characterized on the basis of the data given below.

Dry weight 9.0 g.

Yield w/w 0.90.

NMR (D₂O-DCl) (300 MHz): 7.44-7.45 (s, 5H, ArH) 5.35 (d, 1H-β-lactam)

5.2 (s,1H, CH-Ph), 3.93-(m,1H-β-actam) 2.91-3.03 (s,s, 6H, N(CH₃)₂) 2.55 (m, 2H, CH₂) 2.05 (s, 3H, COCH₃) 1.63 (m, 1H, CH) 1.31 (m, 1H, CH)

Moisture content (by KF) =3.0%

IR (KBr disc)=2980-3660 (s, and broad) 1780, 1700, 1630, 1580, 1460, 1400, 1390, 1380, (m to strong)

### EXAMPLE 2

Preparation of Crystalline Monohydrate of Loracarbef from Mono N, N-DMAc Monohydrate Solvate

Mono N, N-dimethylacetamide monohydrate solvate of loracarbef (10.0 g) was suspended in water (80 ml). 12 N hydrochloric acid (1.0 ml) was added to obtain a clear solution. Activated carbon (1.0 g) was added and the reaction mixture was stirred for 30-40 minutes. The suspension was then filtered and washed with water (30 ml). The temperature of the filtrate was raised to 50-55° C. and the pH was slowly adjusted to 1.8 -1.9 with 8% NH3 solution. The reaction mixture was stirred for 30 minutes at 50-55° C. Stirring was continued for additional 30 minutes and then slowly cooled to 20-25° C. The slurry was washed with water. The cake was dried in air oven at 40-45° C. to yield crystalline loracarbef monohydrate (5.0 g) having bulk density greater than 0.6 g/ml. IR, NMR and X-Ray diffraction pattern of the crystalline loracarbef monohydrate matches with the authentic samples of crystalline loracarbef monohydrate.

### EXAMPLE 3

Preparation of Mono N-methyl Pyrrolidone Monohydrate Solvate of Loracarbef

Step A: Preparation of N-Methyl Morpholine Salt:

To a mixture of N-methyl pyrrolidine (60 ml) and N-methyl morpholine (3.0 g), p-nitrobenzyl 7 β-amino-3-chloro-1-carba (1-dethia)-3-cephem-4-carboxylic acid hydrochloride (10.0 g) was added over 15-20 minutes at −20 to −15° C. The reaction mixture was stirred for 60 minutes.

Step B: Preparation of Mixed Anhydride:

The Na/K Dane salt, 9.5 g (prepared according to the procedure of Dane et al., Angew. Chem., Vol. 74, 873, 1962) was suspended in N-methyl pyrrolidone (120ml) and stirred for 30-35 minutes. The reaction mixture was cooled to −20to −15° C. and methane sulphonic acid (0.15 g) and N-methyl morpholine (0.08 g) were added to it. Ethyl chloroformate (3.3 g) was further added in one portion and stirring was continued for 60-90 minutes at −10 to −15° C.

Step C: Condensation:

N-methylmorpholine hydrochloride solution containing the free amine obtained from Step A was slowly added to the mixed anhydride obtained from Step B at −20° to −10° C. in about 15-20 minutes. The reaction mixture was stirred for 60 minutes. Conc. HCl in H₂O (28 ml in 14 ml H₂O) was added drop wise at −10° to 0° C. to diprotected loracarbef followed by the addition of zinc powder (6.0 g), while maintaining the temperature from 0° to +5° C. The temperature was raised to 20-25° C. and the reaction mixture was stirred for about 60 minutes. Semicarbazide hydrochloride (3.3 g) was added and the stirrng was continued for 30 minutes. The pH of the reaction mixture was adjusted to 2.9 to 3.0 with 28% NH3 solution and then filtered it. The filtrate was washed with N-methyl pyrrolidone (50 ml) and the pH was adjusted to 4.8 to 5.0. The separated solid was further stirred for about 30 minutes and the pH was finally adjusted to 5.8 to 6.2. The reaction mixture was cooled to 20-25° C., acetonitrile (60ml) was added and stirred for another 30 minutes. It was then filtered and the solid was dried under vacuum to give mono N-methyl pyrrolidone monohydrate solvate of loracarbef which was characterized on the basis of the data given below.

NMR (300 MHz) (s): 7.4 (s, 5H, ArH, 5.3 (d, 1H, β-lactam), 5.2 (s, 1H, CH, Ph), 3.83 (m, 1H, β-lactam), 3.3-3.42 (t, 2H, due to N-methyl pyrrolidone), 2.72 (s, 3H, NH₃, due to NMP), 2.46-2.53 (m, 2H, C), 2.32-2.37 (t, 2H, due to NMP), 1.90-1.95 (m, 2H, due to NMP), 1.55 (m, 1H, CH, 1.18-1.22 (m, 1H, CH)

Moisture content (by KF): 5.0% w/w

IR (KBr disc): 2980-3650 (s, and broad) 1780, 1720, 1690, 1600, 1580, 1460, 1400,

### EXAMPLE 4

Preparation of Crystalline Monohydrate of Loracarbef from Mono N-methyl Pyrrolidone Monohydrate Solvate

Loracarbef mono N-methyl pyrrolidone monohydrate solvate (10.0 g) was suspended in water (80 ml). 12 N hydrochloric acid (1.0 ml) was added to obtain a clear solution. Activated carbon (1.0 g) was added and the reaction mixture was stirred for 30-40 minutes. The suspension was then filtered and washed with water (30 ml). The temperature of the filtrate was raised to 50-55° C. and the pH was slowly adjusted to 1.8-1.9 with 8% ammonia solution. The reaction mixture was stirred for 30 minutes at 50-55° C. and the pH was adjusted to 4.5 to 4.8 slowly in 30-35 minutes with stirring at 50-55° C. Stirring was continued for additional 30 minutes and then slowly cooled to 20-25° C. The slurry was washed with water. The cake was dried in air oven at 40-45° C. to yield crystalline loracarbef monohydrate (5.0 g) having bulk density greater than 0.6 g/ml.

IR, NMR and X-Ray diffraction pattern of the crystalline loracarbef monohydrate matches with the authentic samples of crystalline loracarbef monohydrate.

While the present invention has been described in terms of its specific embodiments, certain modifications and equivalents will be apparent to those skilled in the art and are intended to be included within the scope of the present invention.

We Claim:

1. Mono N,N-Dimethylacetamide monohydrate solvate of loracarbef of Formula II-A.

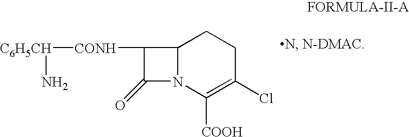

2. The compound of claim 1 which has the following X-ray diffraction powder pattern:

| d | I/Io |
|---|---|
| 15.6 | 17.0 |
| 11.80 | 100 |
| 11.12 | 41 |
| 7.43 | 25 |
| 5.91 | 12 |
| 5.19 | 14 |
| 4.88 | 16 |
| 4.76 | 22 |
| 4.69 | 17 |
| 4.45 | 13 |
| 4.28 | 13 |
| 3.93 | 70 |
| 3.639 | 28 |
| 3.33 | 18 |
| 3.177 | 71 |
| 2.949 | 18 |
| 2.729 | 13 |
| 2.6122 | 13 |

3. Mono N-Methylpyrrolidone monohydrate solvate of loracarbef of Formula II-B.

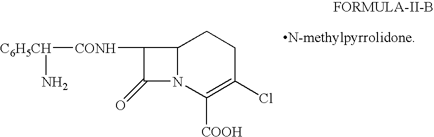

4. The compound of claim 3 which has the following X-ray diffraction powder pattern:

| d | I/Io |
|---|---|
| 15.8248 | 14 |
| 15.2251 | 13 |
| 12.0338 | 100 |
| 8.0954 | 8 |
| 7.5189 | 33 |
| 5.9968 | 13 |
| 5.4668 | 12 |
| 5.3810 | 14 |
| 5.2605 | 18 |
| 4.8863 | 22 |
| 4.7513 | 37 |
| 4.4579 | 21 |
| 4.2997 | 22 |
| 4.1411 | 16 |
| 3.9939 | 55 |
| 3.6421 | 38 |
| 3.3858 | 18 |
| 2.7314 | 15 |

5. A process for the preparation of mono-N, N-dimethylacetamide monohydrate solvate of loracarbef of Formula II-A, 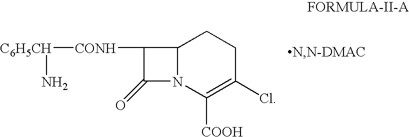
comprising mixing a compound of Formula III, 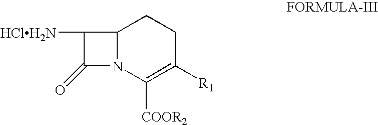
wherein R₁ is hydrogen, trihalo (C₁-C₄ alkyl), C₁-C₄ alkyl, C₁-C₄ substituted alkyl, C₁-C₄ alkoxy, C₁-C₄ substituted alkoxy, C₁-C₆ alkylthio, C₁-C₆ substituted alkylthio, methoxy methyl, carbamoyloxy methyl, acetoxymethyl, C₂-C₆ alkenyl,
C₂-C₆ substituted alkenyl, or halogen such as bromo, chloro, fluoro, and iodo; and
R₂ is a carboxy-protecting group,
with N,N-dimethylacetamide and a base to form a free amine of the compound of formula IV, and 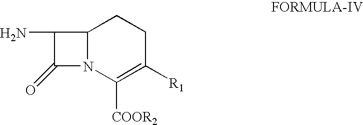
reacting the compound of Formula IV with an acylating agent of Formula V, 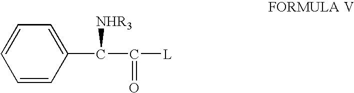
wherein R₃ is an amino protecting group and L is a leaving group.

6. A process for the preparation of mono N-methylpyrrolidone monohydrate solvate of loracarbef of Formula II-B, 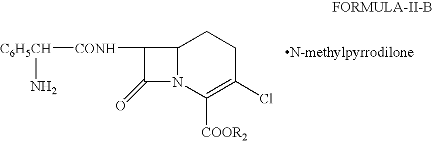
comprising mixing a compound of Formula III, 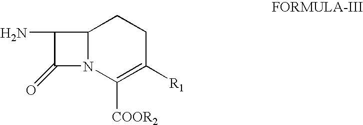
wherein R₁ is hydrogen, trihalo (C₁-C₄ alkyl), C₁-C₄ alkyl, C₁-C₄ substituted alkyl, C₁-C₄ alkoxy, C₁-C₄ substituted aLkoxy, C₁-C₆ aLkylthio, C₁-C₆ substituted alkylthio, methoxy methyl, carbamoyloxy methyl, acetoxymethyl, C₂-C₆ alkenyl, C₂-C₆ substituted alkenyl, or halogen such as bromo, chloro, fluoro, and iodo; and R₂ is a carboxy-protecting group,
with N-methylpyrrolidone and a base, to form a free amine of the compound of formula IV, and 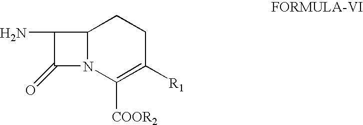
reacting the compound of Formula IV with an acylating agent of Formula V, 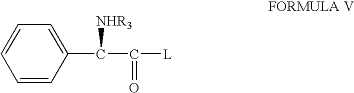
wherein R₃ is an amino protecting group and L is a leaving group.

7. The process according to claim 5 or 6, wherein the carboxyl protecting group, R₂ is selected from the group consisting of allyl, alkyl, benzyl, substituted benzyl, silyl, halo-substituted alkyl and alkoxy alkyl.

8. The process according to claim 7, wherein the carboxyl protecting group is 4-nitrobenzyl.

9. The process according to claim 5 or 6, wherein the amino-protecting group, R₃ is selected from the group consisting of alkoxy-carbonyl, phenoxy-carbonyl, phenoxy-acyl, alkoxy-acyl, aralkoxy-carbonyl, enamino derived from C₁₋₄ alkylacetoacetate and acyl.

10. The process according to claim 5 or 6, wherein the leaving group L is compound of Formula VI, 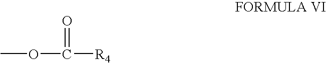
wherein R₄ is selected from the group consisting of halo such as chloro, bromo, iodo or C₁₋₆ alkyl, benzyl, substituted benzyl, phenyl, substituted phenyl, adducts of dicyclohexylcarbodiimide and alkoxy alkyl.

11. The process according to claim 5 or 6, wherein the base is a cyclic amine base containing 0-1 oxygen atom.

12. The process according to claim 11, wherein the cyclic amine base is selected from the group consisting of five- or six- membered tertiary cyclic amines.

13. The process of claim 12, wherein the cyclic amine base is N-methylmorpholine or N-methylpiperazine.

14. A process for the preparation of crystalline monohydrate of loracarbef, the process comprising:
treating mono N,N-dimethylacetamide monohydrate solvate of loracarbef with an acid, and adjusting the pH with a base to afford the crystalline monohydrate of loracarbef.

15. A process for the preparation of crystalline monohydrate of loracarbef, the process comprising:
treating mono N-methylpyrrolidone monohydrate solvate of loracarbef with an acid, and
adjusting the pH with a base to afford the crystalline monohydrate of loracarbef.

16. The process according to claim 14 or 15, wherein the acid used is a mineral acid or an organic acid.

17. The process according to claim 16, wherein the acid is hydrochloric acid.

18. The process according to claim 14 or 15, wherein the base used is ammonia.

19. Crystalline monohydrate of loracarbef having a bulk density greater than or equal to 0.6 g/ml.

20. A pharmaceutical composition comprising:
a therapeutically effective amount of a crystalline monohydrate of loracarbef having a bulk density greater than or equal to 0.6 g/ml;
and one or more pharmaceutically acceptable carriers, excipients or diluents.
